(19) United States
(12) Patent Application Publication
(10) Pub. No.: US 2024/0197172 A1
(43) Pub. Date: Jun. 20, 2024

(54) MYOPIA TREATMENT DEVICE CAPABLE OF IDENTIFYING EYE OPEN AND CLOSED STATES

(71) Applicant: Guanglang (Hainan) Biotechnology Co., Ltd., Haikou (CN)

(72) Inventor: Te Gao, Haikou (CN)

(21) Appl. No.: 18/509,283

(22) Filed: Nov. 14, 2023

(30) Foreign Application Priority Data
Dec. 19, 2022 (CN) 202211633008.2

(51) Int. Cl. A61B 3/113; A61B 3/00

(52) U.S. Cl. CPC A61B 3/113; A61B 3/0008; A61B 3/0025; A61B 2560/04; A61B 2562/02

(57) ABSTRACT

Disclosed is a myopia treatment device capable of identifying eye open and closed states. The myopia treatment device includes a housing, a light source, an optical sensor, and a control module, where the light source is configured to emit a first light ray to an eye area of a user; the optical sensor is configured to acquire light intensity data of the first light ray after the first light ray is reflected, and send the light intensity data to the control module; and the control module is configured to, by comparing the light intensity data sent by the optical sensor with a preset light intensity threshold and a preset time threshold, determine whether the user is in an eye open or closed state. The present disclosure has low cost and low computational complexity in the detection process.

### CROSS REFERENCE TO RELATED APPLICATIONS

The present application claims the benefit of Chinese Patent Application No. 202211633008.2 filed on Dec. 19, 2022, the contents of which are incorporated herein by reference in their entirety.

### TECHNICAL FIELD

The present disclosure relates to a field of eye open and closed states identification, and in particular to a myopia treatment device capable of identifying eye open and closed states.

### BACKGROUND

In recent years, people who have been using electronic devices with screens for a long time may experience symptoms of fatigue or even myopia in their eyes, and the proportion of myopia has been increasing year by year. Therefore, daily prevention and control of myopia is an important technological development direction. Some devices are used to train the eyes for near and distant vision or simulate light for light irradiation therapy of eyeballs, which plays a certain role in the treatment and prevention of myopia.

In the prior art, during light simulation treatment, the eyes of the user are covered by the device, making it hard to identify whether the user is in an eye open or closed state. The user may maintain an eye closed state for a long time rather than to cooperate well with the treatment, thereby affecting the treatment effect. The existing myopia treatment devices mainly utilize an image identification device to identify whether the user is in an eye open or closed state. However, the image processing technology requires high computational performance of the device, requiring the generation of a large amount of graphic data and corresponding processing. The processing is complex and leads to slow response speed, thereby affecting the treatment effect.

### SUMMARY

In order to solve the technical problem of slow response speed, the present disclosure provides a myopia treatment device capable of identifying eye open and closed states.

In order to solve the above technical problem, an embodiment of the present disclosure provides a myopia treatment device capable of identifying eye open and closed states. The myopia treatment device includes a housing, a light source, an optical sensor, and a control module, where

the light source and the control module are arranged inside the housing of the myopia treatment device; the optical sensor is located outside the housing and opposite to eyes of a user; and the control module is electrically connected to the optical sensor;

the light source is configured to emit a first light ray to an eye area of the user;

the optical sensor is configured to acquire light intensity data of the first light ray after the first light ray is reflected, and send the light intensity data to the control module; and

the control module is configured to, by comparing the light intensity data sent by the optical sensor with a preset light intensity threshold and a preset time threshold, determine whether the user is in an eye open or closed state.

In a preferred solution, the optical sensor is a photosensitive sensor; and the preset light intensity threshold includes a first eye open threshold range and a first eye closed threshold range;

the control module is configured to, by comparing the light intensity data sent by the optical sensor with the preset light intensity threshold and the preset time threshold, determine whether the user is in an eye open or closed state, and specifically:

the control module determines whether the light intensity data acquired by the photosensitive sensor falls within the first eye open threshold range;

if the light intensity data falls within the first eye open threshold range, the control module determines that the user is in the eye open state;

if the light intensity data falls within the first eye closed threshold range and a duration thereof reaches a first time, the control module determines that the user is in the eye closed state; and

if the light intensity data falls within the first eye closed threshold range and the duration is less than the first time, the control module determines that the user is in a blinking state.

In a preferred solution, the optical sensor is a photoelectric sensor; and the photoelectric sensor includes an emitting terminal and a receiving terminal; and

the emitting terminal is configured to emit a second light ray to the eye area of the user; and the receiving terminal is configured to acquire light intensity data of the first light ray and the second light ray, and send the light intensity data to the control module.

In a preferred solution, the preset light intensity threshold includes a second eye open threshold range and a second eye closed threshold range;

the control module is configured to, by comparing the light intensity data sent by the optical sensor with the preset light intensity threshold and the preset time threshold, determine whether the user is in an eye open or closed state, and specifically:

the control module determines whether the light intensity data acquired by the photoelectric sensor falls within the second eye open threshold range;

if the light intensity data falls within the second eye open threshold range, the control module determines that the user is in the eye open state;

if the light intensity data falls within the second eye closed threshold range and a duration thereof reaches a second time, the control module determines that the user is in the eye closed state; and

if the light intensity data falls within the second eye closed threshold range and the duration is less than the second time, the control module determines that the user is in a blinking state.

In a preferred solution, the second eye open threshold range and the second eye closed threshold range are set according to the first light ray and the second light ray.

In a preferred solution, the myopia treatment device further includes a speaker connected to the control module; and in responsive to the control module determines that the user is in the eye closed state, the control module controls the speaker to issue an eye closed prompt.

Compared with the prior art, the embodiment of the present disclosure has following beneficial effects.

The embodiment of the present disclosure provides a myopia treatment device capable of identifying eye open and closed states. The myopia treatment device includes a housing, a light source, an optical sensor, and a control module, where the light source and the control module are arranged inside the housing of the myopia treatment device; the optical sensor is located outside the housing and opposite to eyes of a user; the control module is electrically connected to the optical sensor; the light source is configured to emit a first light ray to an eye area of the user; the optical sensor is configured to acquire light intensity data of the first light ray after the first light ray is reflected, and send the light intensity data to the control module; and the control module is configured to, by comparing the light intensity data sent by the optical sensor with a preset light intensity threshold and a preset time threshold, determine whether the user is in an eye open or closed state. Compared to the prior art, the present disclosure compares the light intensity data sent by the optical sensor with the preset light intensity threshold and the preset time threshold, and achieves detection of the eye open and closed states through a difference in the reflectivity of the iris and eyelid skin to light. Compared to the detection method based on image identification and processing in the prior art, the present disclosure has low cost and low computational complexity in the detection process. In addition, the present disclosure simplifies the processing step without the need for extensive processing of image data or feature extraction. Therefore, the present disclosure can achieve fast response and reduce the power consumption of the device.

### BRIEF DESCRIPTION OF THE DRAWINGS

FIG. 1 is a structural diagram of a myopia treatment device capable of identifying eye open and closed states according to an embodiment of the present disclosure;

FIG. 2 is a structural diagram of the myopia treatment device capable of identifying eye open and closed states according to an embodiment of the present disclosure; and

FIG. 3 is a structural diagram of the myopia treatment device capable of identifying eye open and closed states according to an embodiment of the present disclosure.

Reference Signs: 1. housing; 11. light through-hole; 2. light source; 3. photosensitive sensor; 4. eyeball; 5. control module; 6 speaker; 7. photoelectric sensor; 71. emitting terminal; 72. receiving terminal; 8. first light ray; 9. second light ray; and 10. reflected light ray.

### DETAILED DESCRIPTION

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts should fall within the protection scope of the present disclosure.

### Embodiment 1

According to relevant technical records, existing myopia treatment devices are similar to virtual reality (VR) glasses. After the user wears the device, the eye area of the user is covered, making it impossible to observe whether the user is in an eye open or closed state externally.

Among the myopia treatment devices on the market, some lack a device for monitoring the user's eye states, while some use a built-in image identification device and acquire images through an infrared camera or other device. The image processing technology usually involves image acquisition, feature extraction, and identification, and the identification step involves an artificial intelligence (AI) algorithm and requires a large amount of computation. Due to the complex processing, the eye open and closed states identification method based on the image processing technology requires high computational performance of the device and requires a significant amount of computational resources. As a result, problems are arising, such as slow response speed, high hardware requirements, high cost (due to the need for an optimized control module and a specific image acquisition device such as a camera module satisfying a specific requirement), and high power consumption. In addition, the complex technical design may bring extra problems such as large device volume.

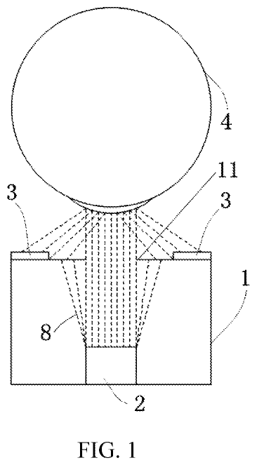

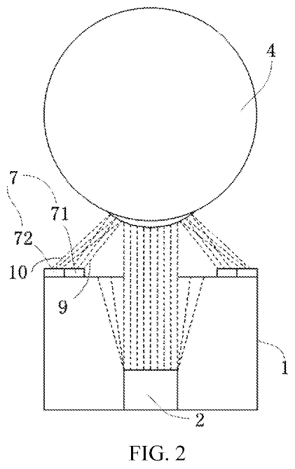

In response to one or more technical problems mentioned above, an embodiment of the present disclosure provides a myopia treatment device capable of identifying eye open and closed states. Referring to FIGS. 1 and 2, the myopia treatment device includes a housing 1, a light source 2, an optical sensor, and a control module 5.

The light source 2 and the control module 5 are arranged inside the housing 1 of the myopia treatment device. The optical sensor is located outside the housing 1 and opposite to eyes of a user. The control module 5 is electrically or communicatively connected to the optical sensor.

The light source 2 is configured to emit a first light ray 8 to an eye area of the user. The first light ray 8 is radiated onto the eye area of the user via a light through-hole 11 of the housing 1, forming a light spot with a diameter of about 10 mm.

The optical sensor is configured to acquire light intensity data of the first light ray 8 after the first light ray is reflected, and send the light intensity data to the control module 5.

The control module 5 is configured to, by comparing the light intensity data sent by the optical sensor with a preset light intensity threshold and a preset time threshold, determine whether the user is in an eye open or closed state.

The control module 5 mainly includes a microcontroller unit (MCU) and an interface device, etc.

In a preferred implementation of this embodiment, referring to FIG. 1, the optical sensor is preferably a photosensitive sensor 3. The preset light intensity threshold includes a first eye open threshold range and a first eye closed threshold range.

The first eye open threshold range can be acquired through a user test. When the user opens his/her eyes, an iris reflects the first light ray 8 emitted by the light source 2. The photosensitive sensor 3 receives a reflected light ray, and acquires a light intensity. A range from −10% of the light intensity to +10% of the light intensity is defined as the first eye open threshold range. The first eye closed threshold range can be acquired through a user test. When the user closes his/her eyes, an eyelid skin reflects the first light ray 8 emitted by the light source 2. The photosensitive sensor 3 receives a reflected light ray, and acquires a light intensity. A range from −10% of the light intensity to +10% of the light intensity is defined as the first eye closed threshold range.

When the tests are conducted to acquire the first eye open threshold range and the first eye closed threshold range, power of the light source 2 is the same as power of the light source 2 in an implementation.

The control module 5 is configured to, by comparing the light intensity data sent by the optical sensor with the preset light intensity threshold and the preset time threshold, determine whether the user is in an eye open or closed state. Specifically:

The control module 5 determines whether the light intensity data acquired by the photosensitive sensor 3 falls within the first eye open threshold range.

If the light intensity data falls within the first eye open threshold range, the control module determines that the user is in the eye open state.

If the light intensity data falls within the first eye closed threshold range and a duration thereof reaches the preset time threshold, that is, a first time, the control module determines that the user is in the eye closed state. The first time can be 1 s, or other duration determined by the specific user.

If the light intensity data falls within the first eye closed threshold range and the duration is less than the first time, the control module determines that the user is in a blinking state. In this preferred implementation, when the user opens his/her eyes, most of the light ray is radiated onto an eyeball 4. The iris on the eyeball 4 absorbs a majority of the light ray, with only a small portion of the light ray being reflected around. The photosensitive sensor 3 can detect the reflected light ray. When the user is in the eye closed state, the light ray is directly radiated onto the eyelid skin. The eyelid skin reflects a majority of the light ray, so the photosensitive sensor 3 can detect a strong light ray. The control module 5 sets a first eye open threshold range for the reflected light intensity in the eye open state and a first eye closed threshold range for the reflected light intensity in the eye closed state. After receiving the light intensity data of the first light ray 8 from the photosensitive sensor 3, the control module compares the light intensity data with the preset threshold range to determine whether the user is in an eye open or closed state.

In another preferred implementation of this embodiment, referring to FIG. 2, the optical sensor is a photoelectric sensor 7. The photoelectric sensor 7 includes an emitting terminal 71 and a receiving terminal 72.

The emitting terminal 71 is configured to emit a second light ray 9 to the eye area of the user. The receiving terminal 72 is configured to acquire light intensity data of a reflected light ray 10, and send the light intensity data to the control module 5. The preset light intensity threshold includes a second eye open threshold range and a second eye closed threshold range.

The second eye open threshold range can be acquired through a user test. When the user opens his/her eyes, the iris reflects the second light ray 9, of an infrared wavelength, emitted by the emitting terminal 71 of the photoelectric sensor 7. The receiving terminal 72 of the photoelectric sensor 7 receives the reflected light ray 10, and acquires a light intensity. A range from −10% of the light intensity to +10% of the light intensity is defined as the second eye open threshold range. The second eye closed threshold range can be acquired through a user test. When the user closes his/her eyes, the eyelid skin reflects the second light ray 9 of the infrared wavelength emitted by the emitting terminal 71 of the photoelectric sensor 7. The receiving terminal 72 of the photoelectric sensor 7 receives the reflected light ray 10, and acquires a light intensity. A range from −10% of the light intensity to +10% of the light intensity is defined as the second eye closed threshold range.

When the tests are conducted to acquire the second eye open threshold range and the second eye closed threshold range, power of the emitting terminal 71 of the photoelectric sensor 7 is the same as power of the emitting terminal 71 of the photoelectric sensor 7 in an implementation.

The control module 5 is configured to, comparing the light intensity data sent by the optical sensor with the preset light intensity threshold and the preset time threshold, determine whether the user is in an eye open or closed state. Specifically:

The control module 5 determines whether the light intensity data acquired by the photoelectric sensor 7 falls within the second eye open threshold range.

If the light intensity data falls within the second eye open threshold range, the control module determines that the user is in the eye open state.

If the light intensity data falls within the second eye closed threshold range and a duration thereof reaches the preset time threshold, that is, a second time, the control module determines that the user is in the eye closed state. The second time can be 1 s, or other duration determined by the specific user.

If the light intensity data falls within the second eye closed threshold range and the duration is less than the second time, the control module determines that the user is in a blinking state.

The second eye open threshold range and the second eye closed threshold range are set according to the first light ray 8 and the second light ray 9. Compared with the above implementation, in this implementation, the settings of the thresholds take into account the superposition of the first light ray 8 and the second light ray 9.

It should be noted that the first light 8 is configured to treat the eyes of the user. In this embodiment, considering a spectral response range of the photoelectric sensor 7, the first light ray 8 and the second light ray 9 are in different wavelength ranges. The second light ray 9 is mainly configured to assist in the detection of the photoelectric sensor 7. The light intensity data of the first light ray 8 and the second light ray 9 can be electrical data. Specifically, when the photosensitive sensor 3 and the photoelectric sensor 7 receive the light ray, a light signal corresponding to the light ray is converted into an electrical signal. In case there is a change in the light intensity, the change will be immediately reflected on the electrical data to cause a corresponding change. By analyzing the change in the electrical signal, the purpose of determining whether the user is in an eye open or closed state can be achieved.

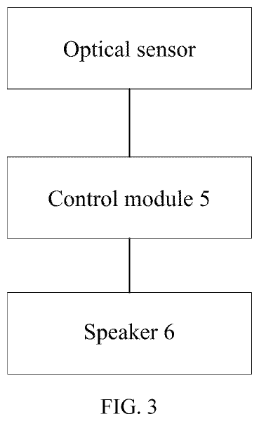

Further, referring to FIG. 3, the myopia treatment device includes a speaker 6. The speaker 6 is connected to the control module 5 electrically or communicatively. In responsive to the control module 5 determines that the user is in the eye closed state, the control module controls the speaker 6 to issue an eye closed prompt to remind relevant personnel around. When the control module determines that the user is in the eye open or blinking state, no prompt is issued.

Compared with the prior art, the embodiment of the present disclosure has following beneficial effects.

The embodiment of the present disclosure provides a myopia treatment device capable of identifying eye open and closed states. The myopia treatment device includes a housing, a light source, an optical sensor, and a control module, where the light source and the control module are arranged inside the housing of the myopia treatment device; the optical sensor is located outside the housing and opposite to eyes of a user; the control module is electrically connected to the optical sensor; the light source is configured to emit a first light ray to an eye area of the user; the optical sensor is configured to acquire light intensity data of the first light ray after the first light ray is reflected, and send the light intensity data to the control module; and the control module is configured to, by comparing the light intensity data sent by the optical sensor with a preset light intensity threshold and a preset time threshold, determine whether the user is in an eye open or closed state. Compared to the prior art, the present disclosure compares the light intensity data sent by the optical sensor with the preset light intensity threshold and the preset time threshold, and achieves detection of the eye open and closed states through a difference in the reflectivity of the iris and eyelid skin to light. Compared to the detection method based on image identification and processing in the prior art, the present disclosure has low cost and low computational complexity in the detection process. In addition, the present disclosure simplifies the processing step without the need for extensive processing of image data or feature extraction. Therefore, the present disclosure can achieve fast response and reduce the power consumption of the device.

The objectives, technical solutions, and beneficial effects of the present disclosure are further described in detail through the above specific embodiments. It should be understood that the above are merely some specific embodiments of the present disclosure, but are not intended to limit the protection scope of the present disclosure. It should be particularly noted that, any modifications, equivalent substitutions, improvements, and the like made by those skilled in the art within the spirit and principle of the present disclosure should be included within the protection scope of the present disclosure.

1. A myopia treatment device capable of identifying eye open and closed states, comprising a housing, a light source, an optical sensor, and a control module, wherein
the light source and the control module are arranged inside the housing of the myopia treatment device; the optical sensor is located outside the housing and opposite to eyes of a user; and the control module is electrically connected to the optical sensor;
the light source is configured to emit a first light ray to an eye area of the user;
the optical sensor is configured to acquire light intensity data of the first light ray after the first light ray is reflected, and send the light intensity data to the control module; and
the control module is configured to, by comparing the light intensity data sent by the optical sensor with a preset light intensity threshold and a preset time threshold, determine whether the user is in an eye open or closed state.

2. The myopia treatment device capable of identifying eye open and closed states according to claim 1, wherein the optical sensor is a photosensitive sensor; and the preset light intensity threshold comprises a first eye open threshold range and a first eye closed threshold range;
the control module is configured to, by comparing the light intensity data sent by the optical sensor with the preset light intensity threshold and the preset time threshold, determine whether the user is in an eye open or closed state, and specifically:
the control module determines whether the light intensity data acquired by the photosensitive sensor falls within the first eye open threshold range;
if the light intensity data falls within the first eye open threshold range, the control module determines that the user is in the eye open state;
if the light intensity data falls within the first eye closed threshold range and a duration thereof reaches a first time, the control module determines that the user is in the eye closed state; and
if the light intensity data falls within the first eye closed threshold range and the duration thereof is less than the first time, the control module determines that the user is in a blinking state.

3. The myopia treatment device capable of identifying eye open and closed states according to claim 1, wherein the optical sensor is a photoelectric sensor, and the photoelectric sensor comprises an emitting terminal and a receiving terminal; and
the emitting terminal is configured to emit a second light ray to the eye area of the user; and the receiving terminal is configured to acquire light intensity data of the first light ray and the second light ray, and send the light intensity data to the control module.

4. The myopia treatment device capable of identifying eye open and closed states according to claim 3, wherein the preset light intensity threshold comprises a second eye open threshold range and a second eye closed threshold range;
the control module is configured to, by comparing the light intensity data sent by the optical sensor with the preset light intensity threshold and the preset time threshold, determine whether the user is in an eye open or closed state, and specifically:
the control module determines whether the light intensity data acquired by the photoelectric sensor falls within the second eye open threshold range;
if the light intensity data falls within the second eye open threshold range, the control module determines that the user is in the eye open state;
if the light intensity data falls within the second eye closed threshold range and a duration thereof reaches a second time, the control module determines that the user is in the eye closed state; and
if the light intensity data falls within the second eye closed threshold range and the duration thereof is less than the second time, the control module determines that the user is in a blinking state.

5. The myopia treatment device capable of identifying eye open and closed states according to claim 4, wherein the second eye open threshold range and the second eye closed threshold range are set according to the first light ray and the second light ray.

6. The myopia treatment device capable of identifying eye open and closed states according to claim 2, wherein the myopia treatment device further comprises a speaker connected to the control module; and in responsive to the control module determines that the user is in the eye closed state, the control module controls the speaker to issue an eye closed prompt.

7. The myopia treatment device capable of identifying eye open and closed states according to claim 4, wherein the myopia treatment device further comprises a speaker connected to the control module; and in responsive to the control module determines that the user is in the eye closed state, the control module controls the speaker to issue an eye closed prompt.
